(19) United States
(12) Patent Application Publication
(10) Pub. No.: US 2003/0216695 A1
(43) Pub. Date: Nov. 20, 2003

(54) Needle syringe

(76) Inventor: Chang-Ming Yang, Miaoli (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/350,067

(22) Filed: Jan. 24, 2003

(30) Foreign Application Priority Data
May 17, 2002 (TW) 091110452
Jul. 3, 2002 (TW) 091114695
Jul. 31, 2002 (TW) 091117118
Oct. 25, 2002 (TW) 091124892

(51) Int. Cl. A61M 5/24

(52) U.S. Cl. 604/200; 604/141; 604/133

(57) ABSTRACT

A needle syringe, which uses an air compressor to generate suction force to withdraw the air in the syringe and form vacuum suction to withdraw drugs or blood into the syringe to attain the objective of withdrawal. Besides, the compressed air by the air compressor can be charged into the syringe to generate push force to inject drugs or blood in the syringe into patient body or a blood storage test tube. Through pressing the blocking section of the needle socket, the elastic fixture in the blocking section is loosen off to allow the needle socket along with the needles retracting into the syringe body.

### BACKGROUND OF THE INVENTION

1. Field of the Invention

The present invention is related to a needle syringe, which especially refers to the type that uses compressed air or vacuum suction for blood withdrawal or drug injection and has the needle retractable into the syringe after use.

2. Description of the Prior Art

Traditional needle injectors have several structural features. On top of syringe, there is a needle connector that connects to an injection needle. Inside syringe, there is a piston plunger, which is pulled outward to create negative pressure, so blood and drugs can be withdrawn into the syringe. Then, the piston plunger is pushed into the syringe to inject blood or drugs through nozzle. However, such traditional needle syringes are operated manually through pulling or pushing the piston plunger, so vacuum suction or push force is generated for injection or withdrawal. As a result, manual pull or push may cause the withdrawal or injection process too fast or too slow due to uneven forces to pull or push the piston plunger, so the patients have numb feeling, discomfort or pain. Besides, due to inadvertent operation by medical staffs, patients have hematoma where the operation is applied. In addition, over-sampling or under-sampling may occur in blood withdrawal operation. Further, because the injection needles are installed on such traditional syringes, medical staffs need to be very careful when they are handling the used syringes to avoid needle sticks or bacteria infection. Apparently, the above-mentioned traditional syringes still have many disadvantages and need further improvement.

In view of all kinds of disadvantages for the traditional needle syringes, the inventor for the present invention was eager to make improvement and innovation. After many years of dedicated research and development, the needle syringe in the present invention is successfully developed.

### SUMMARY OF THE INVENTION

The objective for the present invention is to complete injection operation by using an air compressor to force a balloon inside the syringe body to push drugs or withdraw blood.

Another objective for the present invention is to use an air compressor to suck air from the balloon inside the syringe body and create vacuum suction for withdrawal operation.

Another objective for the present invention is to provide a mechanism that the needle can be retracted into the syringe by releasing the elastic element inside a blocking section that pushes the bottom of needle adapter.

The needle syringe to attain the above objectives will comprise:

a syringe body, at one end of which there is a needle connector, at inner edge of which there is a retainer, while at the other end of the syringe there is an opening;

a needle socket, which is installed inside the syringe and at the rear of which there is a blocking section, inside which there is a holding space for an elastic fixture;

a balloon, which is made of ductile material in long stripe and installed at outer edge of the rear of syringe;

a cylindrical plug, which is made of elastic material in cylindrical shape and inserted at the rear opening of syringe;

a nozzle, at the front of which there is a socket with several vent holes so the nozzle can pass through the cylindrical plug into a balloon, while at the rear of the nozzle there is a receptacle; and

a transport tubing, one end of which is installed into the receptacle of nozzle while the other end of which connects to an air compressor.

For the present invention, an air compressor is used to suck air from the balloon, so vacuum suction is generated inside the syringe to withdraw drugs or blood into the syringe; or an air compressor is used to charge air into the balloon, so the balloon injects the drugs or blood in the syringe into patient's body or blood storage tubes.

### BRIEF DESCRIPTION OF THE DRAWINGS

The drawings disclose an illustrative embodiment of the present invention that serves to exemplify the various advantages and objects hereof, and are as follows:

FIG. 1 is an illustration of a three-dimensional view of the syringe for the present invention.

FIG. 2 is a three-dimensional disassembling diagram for the syringe in the present invention.

FIG. 3 is a side view for the action of the syringe (1).

FIG. 4 is a side view for the action of the syringe (2).

FIG. 5 is a side view for the action of the syringe (3).

FIG. 6 is a side view for the action of the syringe (4).

FIG. 7 is a cross-sectional view for the needle socket.

FIG. 8 is a side view for the action of the needle socket.

FIG. 9 is a side view for the syringe in the first embodiment.

FIG. 10 is a side view for the needle syringe in the second embodiment;

FIG. 11 is a side view for the elastic fixture.

FIG. 12 is a side view for the action of the elastic fixture.

FIG. 13 is a side view for the elastic fixture in the first embodiment.

FIG. 14 is a side view for the action of the elastic fixture in the first embodiment.

FIG. 15 is a side view for the elastic fixture in the second embodiment.

FIG. 16 is a side view for the action of the elastic fixture in the second embodiment.

FIG. 17 is a side view for the needle socket in the first embodiment.

FIG. 18 is a side view for the action of the needle socket in the first embodiment (1).

FIG. 19 is a side view for the action of the needle socket in the first embodiment (2).

FIG. 20 is a side view for the needle socket in the second embodiment.

FIG. 21 is a side view for the action of the needle socket in the second embodiment.

FIG. 22 is a side view for the needle socket in the third embodiment.

FIG. 23 is a side view for the action of the needle socket in the third embodiment (1).

FIG. 24 is a side view for the action of the needle socket in the third embodiment (2).

FIG. 25 is a side view for the needle socket in the fourth embodiment.

FIG. 26 is a side view for the needle socket in the fifth embodiment.

FIG. 27 is a side view for the needle socket in the sixth embodiment.

FIG. 28 is a side view for the needle socket in the seventh embodiment.

FIG. 29 is a side view for the action of the needle socket in the seventh embodiment (1).

FIG. 30 is a side view for the action of the needle socket in the seventh embodiment (2).

FIG. 31 is a side view for the needle socket in the eighth embodiment.

FIG. 32 is a side view for the needle socket in the ninth embodiment;

FIG. 33 is the layout for the air compressor.

FIG. 34 is a side view for the nozzle in another embodiment.

FIG. 35 is a side view for the needle syringe in the third embodiment;

FIG. 36 is a side view for the action of the needle syringe in the fourth embodiment (1);

FIG. 37 is a side view for the action of the needle syringe in the fourth embodiment (2);

FIG. 38 is a side view for the needle syringe in the fifth embodiment;

FIG. 39 is a three-dimensional disassembling diagram for the piston and the plunger in the first embodiment;

FIG. 40 is a three-dimensional disassembling diagram for the piston and the plunger in the second embodiment;

FIG. 41 is a three-dimensional disassembling diagram for the piston and the plunger in the third embodiment;

FIG. 42 is an illustration for the action of the piston and the plunger in the third embodiment;

FIG. 43 is a three-dimensional view for the needle syringe in the sixth embodiment;

FIG. 44 is a three-dimensional view for the needle syringe in the seventh embodiment;

FIG. 45 is a three-dimensional view for the needle syringe in the eighth embodiment;

FIG. 46 is a three-dimensional view for the syringe body of the needle syringe in the first embodiment;

FIG. 47 is a three-dimensional view for the syringe body of the needle syringe in the second embodiment;

FIG. 48 is a three-dimensional view for the syringe body of the needle syringe in the third embodiment;

FIG. 49 is a side view for the needle socket in the tenth embodiment;

FIG. 50 is a side view for the needle socket in the eleventh embodiment;

FIG. 51 is a side view for the action of the needle syringe in the ninth embodiment (1);

FIG. 52 is a side view for the action of the needle syringe in the ninth embodiment (2);

FIG. 53 is a side view for the action of the needle syringe in the ninth embodiment (3);

FIG. 54 is the cross-sectional view for the 10^{th} embodiment for the needle syringe.

### DETAILED DESCRIPTION OF THE PREFERRED EMBODIMENT

For the present invention, the needle syringe uses air compressor to suck air from the syringe or charge air into the syringe, so push force or suction force is generated to enable the action of withdrawal or injection.

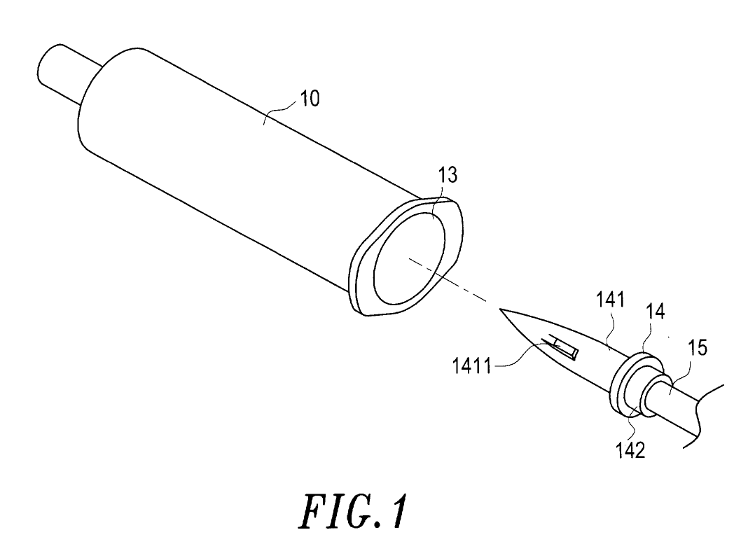

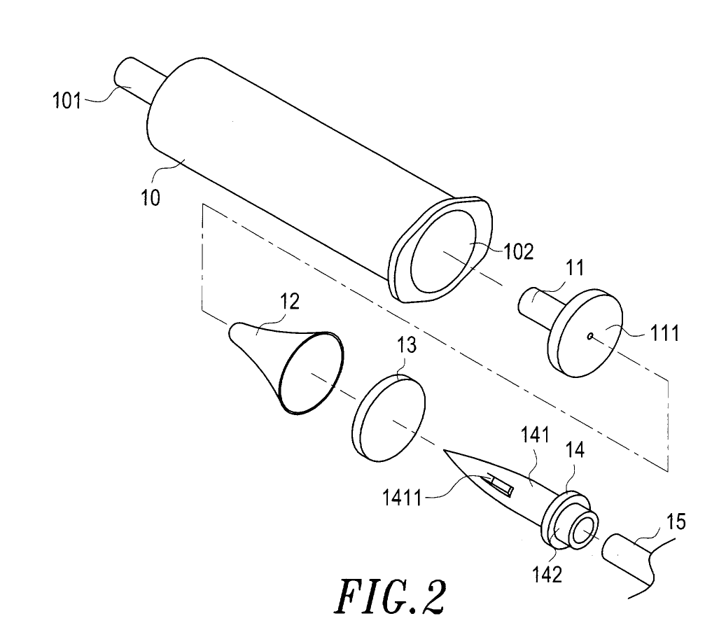

Please refer to FIGS. 1 and 2. The needle syringe for the present invention mainly comprises:

a syringe body 10, at one end of which there is a needle connector 101, at the inner edge of which there is a retainer 1011, while the other end of the syringe body 1 there is an opening 102;

a needle socket 11, which is installed in the syringe body 10 and the front of which is available for needle placement, while the rear of which there is a blocking section 111, inside which there is a holding space 112 for an elastic fixture 113, which is composed of two interlocking pushing stoppers 1131, on the extension of one of which there is a bulging block 20, which extends along the outer edge of blocking section 111 and holds to the inner edge of the syringe body 10, with two pushing stoppers 1131 stuck by adhesive 1132;

a balloon 12, which is made of ductile material in a stripe;

a cylindrical plug 13, which is made of elastic materials in a cylindrical shape;

a nozzle 14, the front of which has a socket 141, which has several vent holes 1411, and the rear of which has a receptacle 142; and

a transport tubing 15, one end of which is installed in the receptacle 142 of a nozzle 14 and the other end of which connects to air compressor;

the balloon 12 is installed at the outer edge of the syringe body 10 with a cylindrical plug 13 inserted into an opening 102 at the rear end of the syringe body 10, so with the exception of the part installed at outer edge of the syringe body 10, all the remaining part of the balloon 12 is pushed into the syringe body 10 by the cylindrical plug 13.

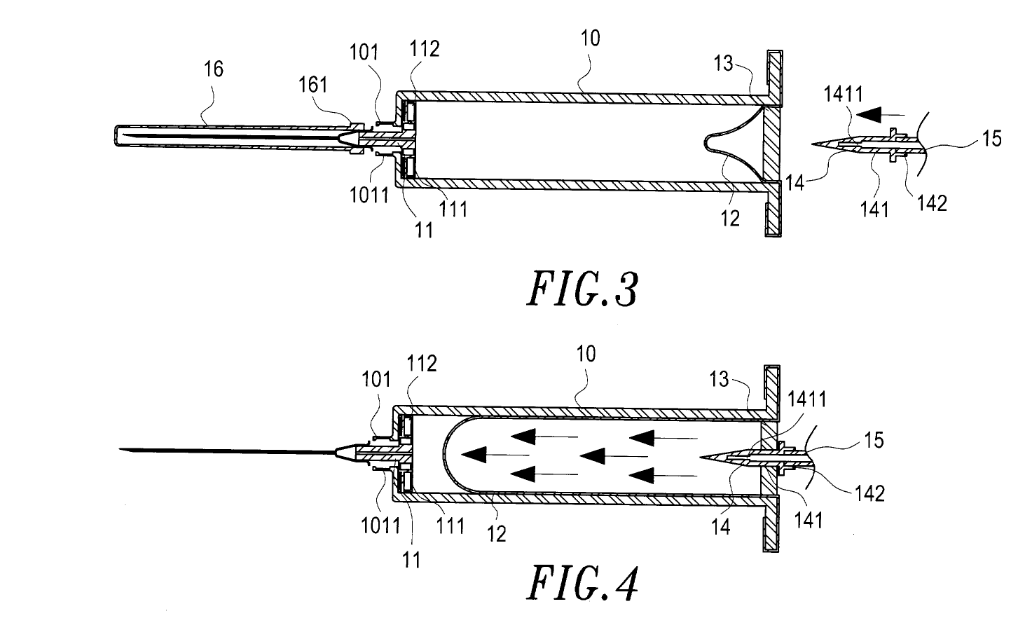

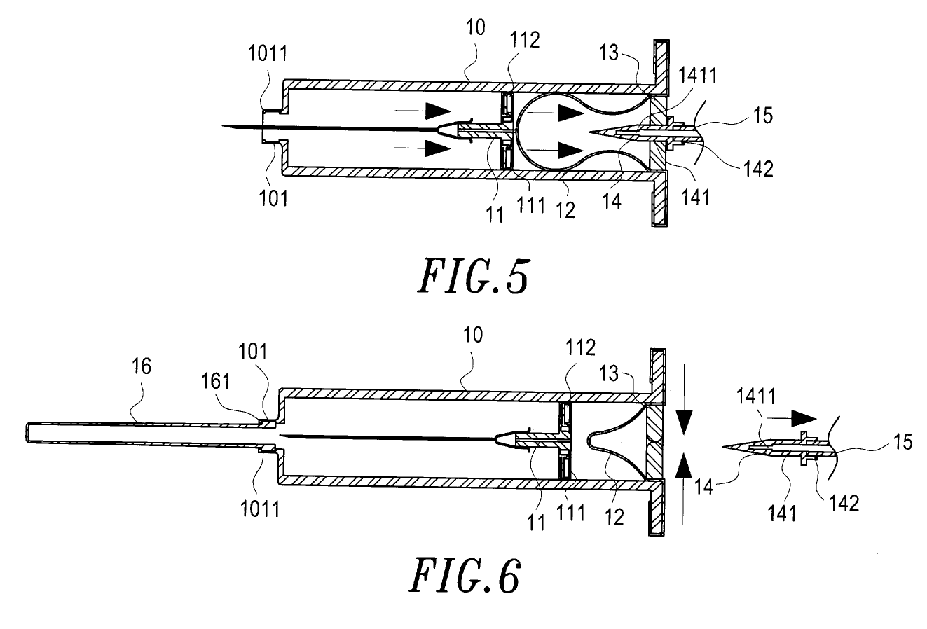

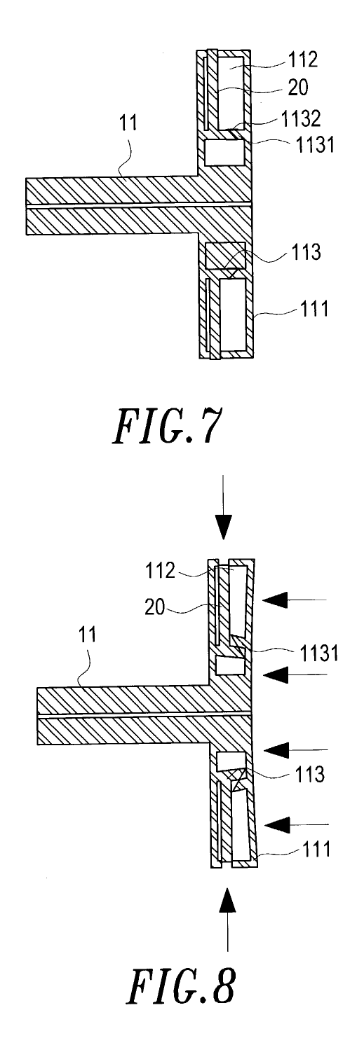

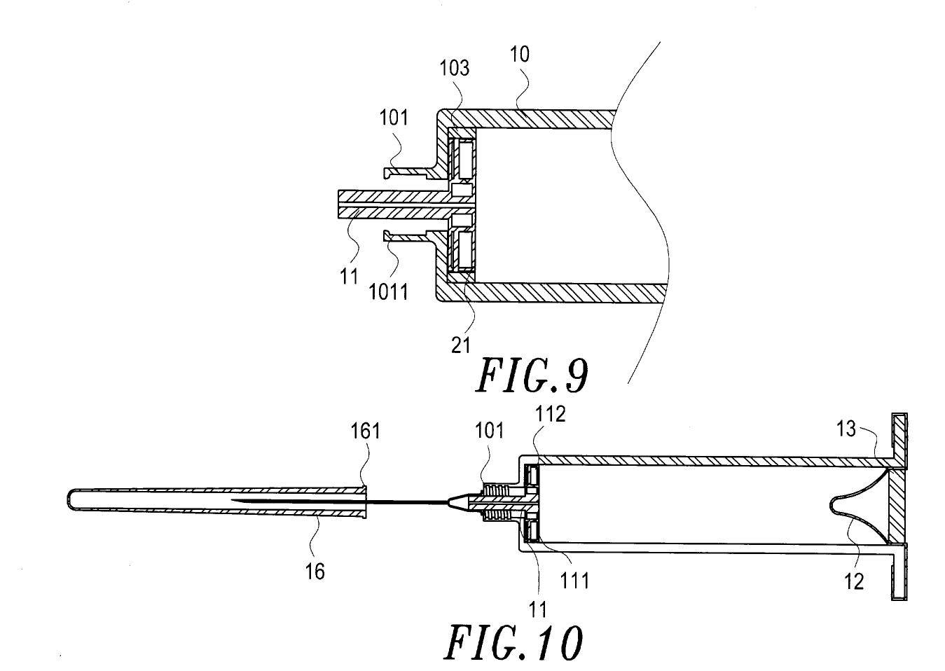

Please refer to FIGS. 3, 4, 5, 6, 7, 8, 9 and 10. When the needle syringe for the present invention is in use, first the injection needle and the needle cap 16 are installed on the needle socket 11, and then after removal of the needle cap 16 the nozzle 14 uses the front socket 141 for the cylindrical plug 13 to pass through and into the balloon 12. Afterward, the air compressor charges air through the transport tubing 15 and the vent holes 1411 on the nozzle 14 into the balloon 12 of the syringe body 10. So the balloon 12 is inflated to tightly adhere to the inner wall of the syringe body 10. The needle in the needle socket 11 is inserted into a drug bottle or the blood vessel of a patient. Meanwhile, the air compressor is activated to suck out the air in the balloon 12 through vent holes 1411 on the nozzle 14 and the transport tubing 15. Immediately, vacuum suction is generated in the syringe body 10 to suck the drugs or blood into the space formed by the syringe body 10 and the balloon 12. To this point, the action for withdrawing drugs or blood is completed. The following is to insert the needle into the patient body or the blood storage tube. With air compressor to charge air through the transport tubing 15 and the nozzle 14 into the balloon 12 of the syringe body 10. The balloon 12 is therefore inflated to push the drugs or blood existing in the space formed by the balloon 12 and the syringe body 10 into the patient body or blood storage tube through the needle in the needle socket. In the meantime, the air compressor continues to charge air into the balloon 12, which then continues inflation to pressure the blocking section 111 of the needle socket 11. When external forces press the two interlocking pushing stoppers 1131 in the blocking section 111, they stick the adhesive and become mutually interlocked. The bulging block 20 in the pushing stopper 1131 retracts into the holding space 112 in the blocking section 111 so the interfering force on the needle socket 11 and the inner wall of the syringe body disappears. The needle socket 11 and the needle retract into the syringe body and are locked in the retainer 1011 of the needle connector 11 through a circular bulging block 161 at the outer edge of the needle cap 16. The needle cap 16 is fixed onto the syringe body 10. After the nozzle 14 is removed from the cylindrical plug 13, the hole left on the cylindrical plug 13 will be sealed up by itself due to material elasticity. Further, a circular elastic element 12 can be added between the syringe body 10 and the blocking section 111 of the needle socket 11. The elastic element 103 is used to increase the resistance of the blocking section 111. Optionally, the retainer 1011 of the needle connector 11 is designed with screw threads so the needle cap 16 is screwed with the syringe body 10, Moreover, on the interior fringe of the soft cork 101, threads of the screw may be fixed thereon for the circular bulging part 161 of the soft cork 101 to screw in.

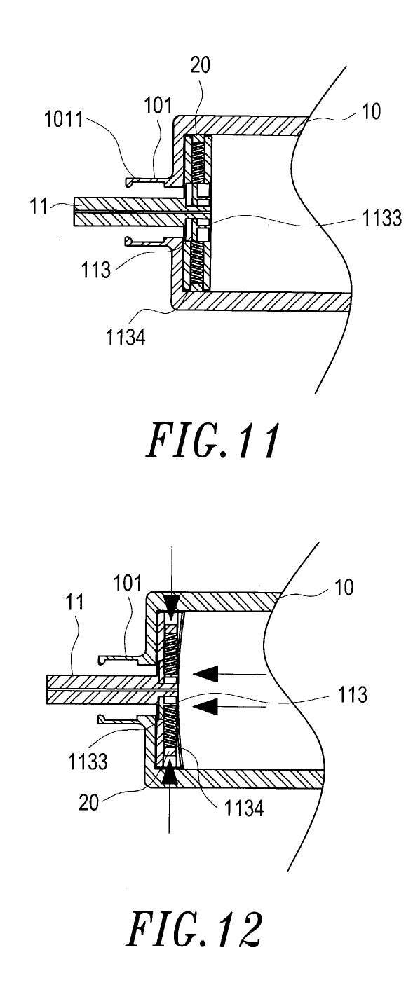

Please refer to FIGS. 11 and 12. The elastic fixture 113 can also be composed of one or more than one T stoppers 1133 and several springs 1134. One end of the spring 1134 has a bulging block 20, which extends along the outer edge of the blocking section 111 and holds onto the inner wall of the syringe body 10. The other end of the spring 1134 holds onto the extended longer side of the T stopper 1133. A groove 1121 at the side of the holding space 112 is available for the placement of the extended longer side of the T stopper 1133. When the balloon 12 pushes the elastic fixture 113, the extended longer side of the T stopper 1133 is pushed into the groove 1121. Therefore, the spring 1134 along with the bulging block 20 retracts into the holding space 112. At the moment, the interfering force between the bulging block 20 and the inner wall of the syringe body disappears to allow the needle socket 11 retracting into the syringe body.

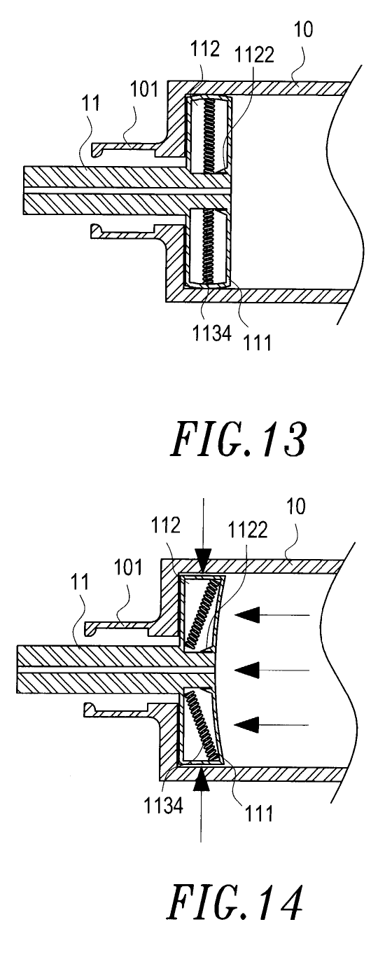

Please refer to FIGS. 13 and 14. The elastic fixture 113 can be held onto the holding space 112 of the blocking section 111 by several springs 1134. So the two sides of the blocking section 111 support the inner wall of the syringe body 10. Besides, there is a push block 1122 for one side of each spring 1134 in the holding space 112. When the balloon 12 pushes the elastic fixture 113, each push block 1122 will separate each spring 1134. As a result, the interfering force between the spring 1134 and the syringe body disappears and the needle socket 11 is retracted into the syringe body.

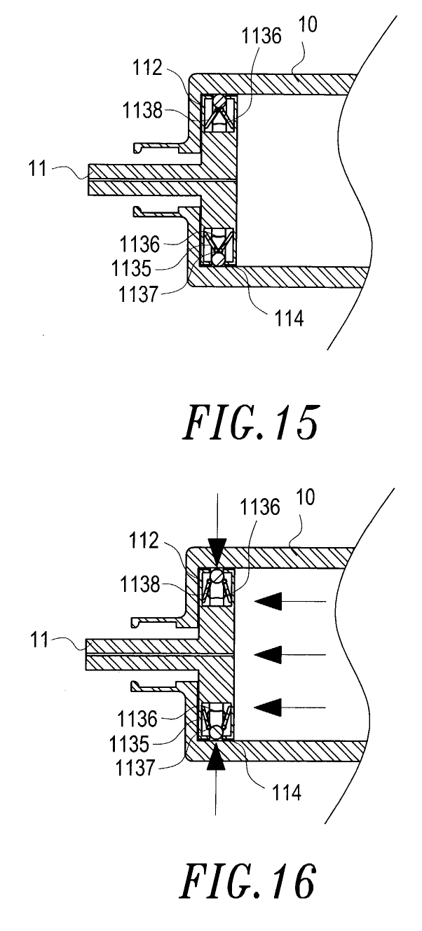

Please refer to FIGS. 15 and 16. The elastic fixture 113 can also be composed of several clamp modules 1135 and spring coils 114 made of materials like rubber. The clamp module 1135 is composed of triangular elastic plate 1136 that is made of bent elastic material. The two ends of the opening 102 of the triangular elastic plate 1136 are bent into a circular clamping section 1137, inside which there is a lever 1138 that extends to the outer edge of the triangular elastic plate 1136. While the bottom of the triangular plate 1136 forms an elastic arch structure. In addition, the outer edge of the spring coil 114 holds onto the inner wall of the syringe body 10. While the inner edge of the spring coil holds onto the clamping section 1137 of the clamp module 1135. When the balloon 312 pushes the elastic fixture 113, the lever 1138 is forced to open the two clamping sections 1137 of the triangular elastic plates 1136. Therefore, the elastic spring coils 114 retract into the opening of the triangular elastic plate 1136. As a result, the interfering force on the spring coils 114 and the inner wall of the syringe body disappears.

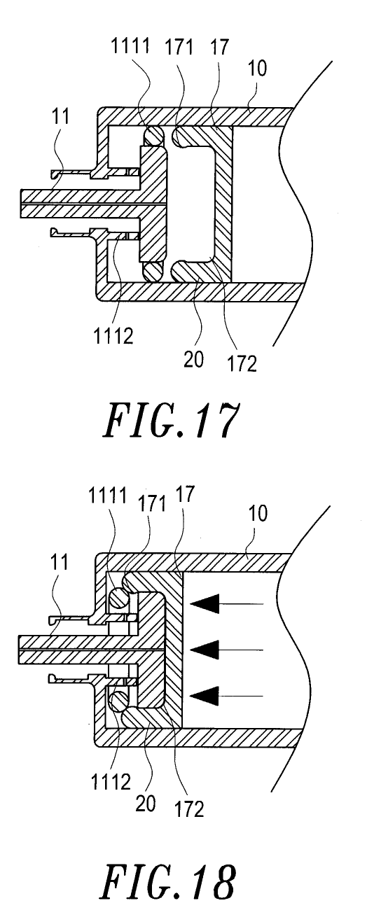

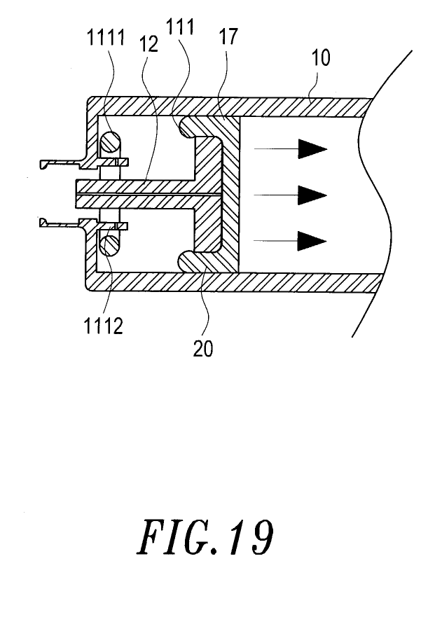

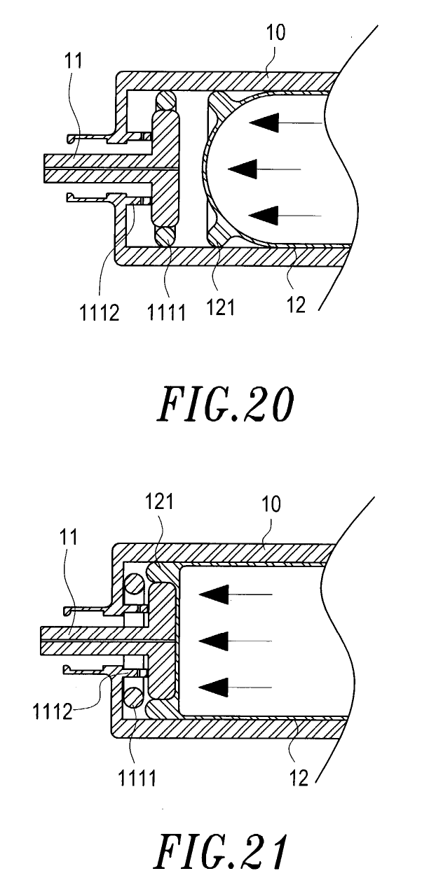

Please refer to FIGS. 17, 18, 19, 20 and 21. The blocking section 111 of the needle socket 11 may not need the holding space 112 for the elastic fixture 113. At the outer edge of the blocking section 111 there are spring coils 1111 made of elastic materials like rubber. Besides, one or more than one supports 1112 are installed between the syringe body 10 and the blocking section 111. A piston 17 is installed in the syringe body 10. A bulging block 20 is installed and surrounds the piston 17 at the position corresponding to the spring coil 1111 and forms a clamping section 172 at the position between the piston and the bulging block 20. Besides, the inner edge of the bulging block 20 top has a spherical bulging block 171. When the piston 17 is pushed by air, the bulging block 20 of the piston 17 pushes the spring coils off the outer edge of the blocking section 111 so the blocking section 111 is placed in the clamping section 172. In the meantime, the spherical bulging part 171 surrounding the bulging block 20 is hooked into the front of the blocking section 111, so the blocking section 111 of the needle socket 11 is covered by the bulging block 20 in the clamping section 172. The piston 17 is pulled back by the negative pressure to allow the needle socket 11 and the syringe needle retracting into the syringe body 10. Further, the piston 17 can be replaced by the balloon 12, which front has a circular bulging part 121 to surround the balloon 12. When the balloon 12 is inflated and presses the blocking section 111 of the needle socket 11, the circular bulging part 121 pushes the spring coils 1111 off the outer edge of the blocking section 111. Immediately, the resisting force formed through the spring coils 1111 between the blocking section 111 and the syringe body 10 disappears to allow the needle socket 11 and the needle retracting into the syringe body. Alternatively, the spring coil can be made of non-elastic material.

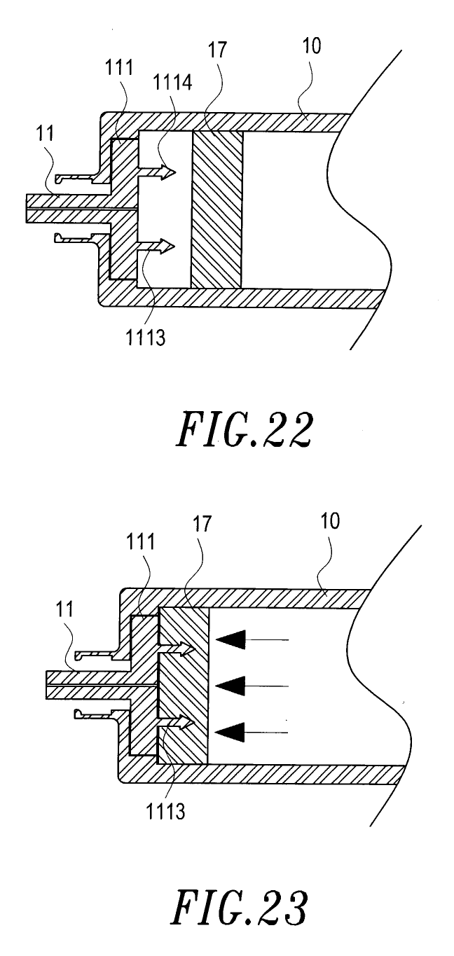

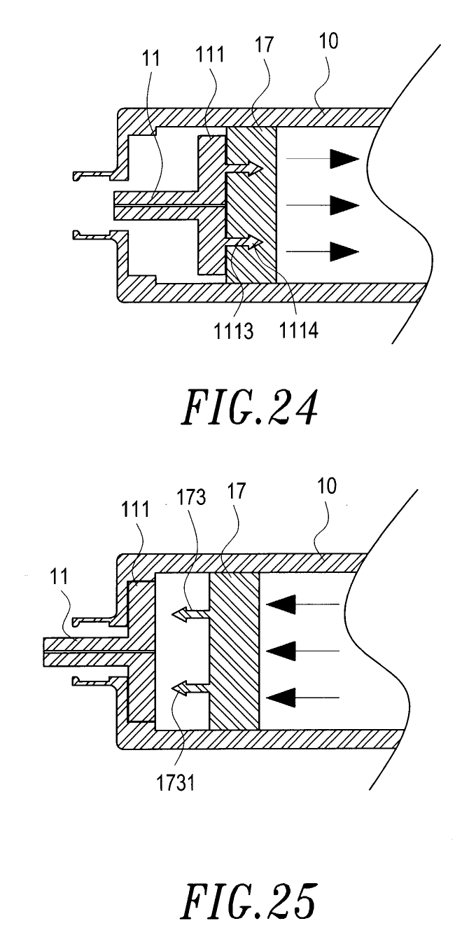

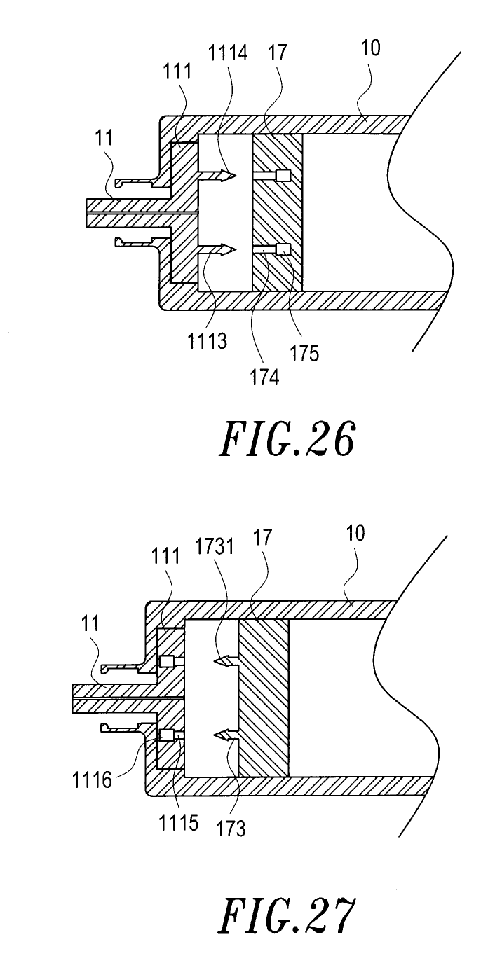

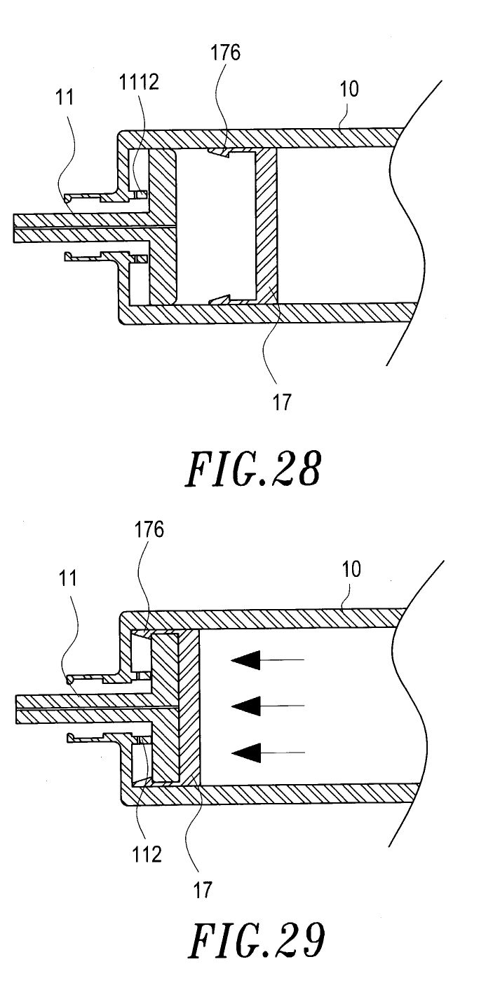

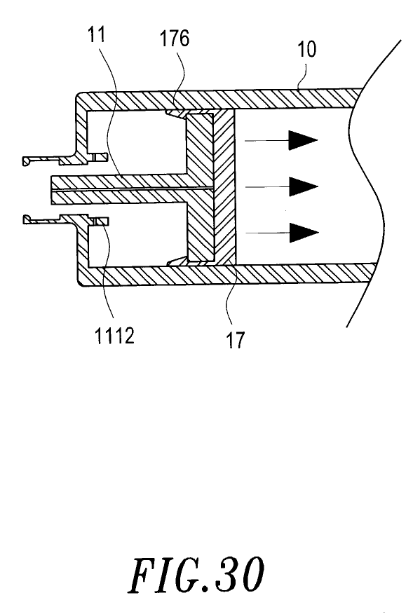

Please refer to FIGS. 22, 23, 24, 25, 26 and 27. The rear of the blocking section 111 has several locking tenons 1113 with reversed hooks 1114. There is a piston 20 of elastic material in the syringe body 10. When the piston 17 is pushed forward by air, the locking tenon 1113 of the blocking section 111 will enter the piston 17 or pass through the piston 17. Meanwhile, the hole punched by the locking tenon 1113 on the piston 17 is sealed by itself due to the elasticity and wraps around the locking tenon 1113. The reversed hook 1114 of the locking tenon 1113 locks the piston 17 and the blocking section 111. Then, the piston 17 is pulled out by negative pressure, so the needle socket 11 and needle are pulled into the syringe body 10. Besides, the blocking section 111 can also be made of elastic materials. The piston 17 has several locking tenons 173 with reversed hooks 1731 across the blocking section 111. The piston 17 may be made of non-elastic materials too. The piston 17 has several guiding holes 174 of corresponding quantity and at corresponding position to the locking tenons 1113. Besides, at the bottom of each guiding hole 174 there is a holding groove wider than the guiding hole 174. When the piston 17 is pressed by air, the locking tenon 1113 on the blocking section 111 is forced to enter the holding groove 175 through the guiding hole 174 of the piston 17. The reversed hooks 1114 for each locking tenon 1113 hold onto the wall of the holding groove 175, so the piston 17 combines with the blocking section 171. Then the piston 17 along with the needle socket 11 and the needle is pulled into the syringe body 10. Further, when the locking tenon 173 is installed on the piston 17, the blocking section 111 can be made of non-elastic materials. Besides, several guiding holes 1115 are made for the blocking section 111 in corresponding quantity and at the corresponding position to the locking tenon 173 of the piston 17. At the bottom of each guiding hole 1115 there is a holding groove 1116 wider than the guiding hole 1115. Further, a pathway can be made in the holding groove 175 or 1116 for the above-mentioned piston 17 or blocking section 111 to connect to the needle socket 11 as passage of drugs.

Please refer to 28, 29 and 30. The blocking section 111 can also be made of elastic materials. The outer edge of the blocking section 111 connects to the inner wall of the syringe body 10. The balloon 12 is replaced by a piston 17. The outer edge of the piston 17 corresponding to the blocking section 111 has surrounding locking hooks 176. When the piston 17 is pushed by air, because the blocking section 111 is made of elastic materials, the locking hooks 176 of the piston 17 can push the blocking section 111 toward the syringe body 10 through the contact surface on the blocking section 111 and the inner wall of the syringe body 10. As a result, the locking hooks 176 pass through the outer edge of the blocking section 111 and lock onto the blocking section 111. Negative pressure is then used to pull the piston 17 backward. In the meantime, the locking hooks 176 is locked onto the blocking section 111. Hence, the piston 17 moves the needle socket 111 with the injection needle into the syringe body 10. Further, a pathway can be open in the holding groove on the above-mentioned piston 17 or blocking section 111 to connect to the needle socket 11 as drug passage.

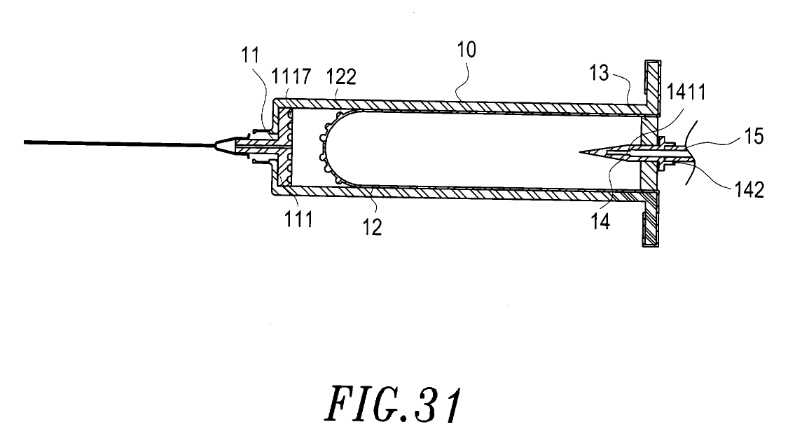

Please refer to FIG. 31. The blocking section 111 of the needle socket 11 may not need the holding space 112 for the elastic fixture 113. Instead, the rear of the blocking section 111 has several circular grooves 1117. In addition, the outer edge of the balloon 12 has several spherical bulging parts 122 at the position corresponding to the circular grooves 1117.

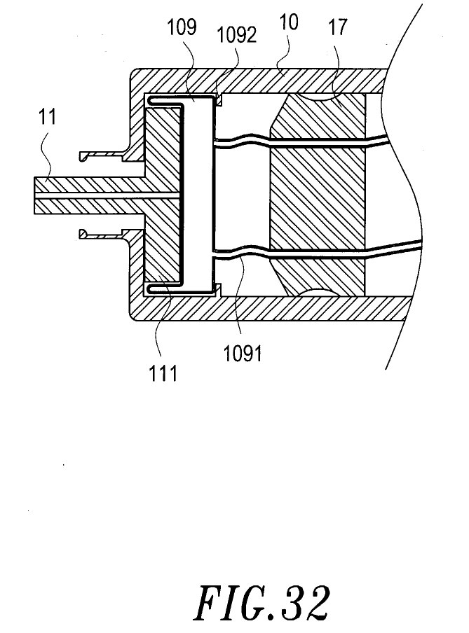

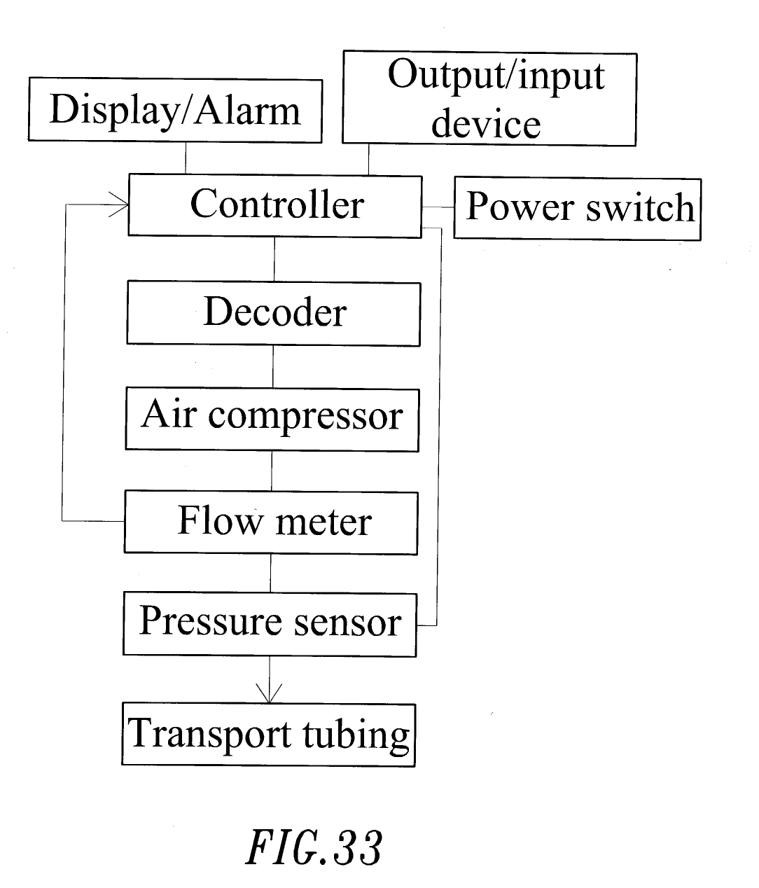

Please refer to FIG. 32. The said retainer 111 has a holding space 112 for elastic fixation structure 113. The rear of the retainer 111 has an air bag 109 made of balloon material. The outer edge of the air bag extends to cover the outer side retainer 111. The rear of the air bag 109 has one or more than one air conduits 1091 made of elastic materials. Besides, on the inner wall of the syringe body 10, there are several bulging parts 1092, which block the rear of the air bag 109 so the air bag 109 will not be pulled backward by the elastic air conduit 1091.

Please refer to FIG. 33. There are a display and an alarm on the air compressor. When injection and withdrawal are completed or clogged injection outlet or leakage is found, the alarm will sound to notify the medical staffs. Besides, the air compressor has a controller, a decoder and a flow meter to allow adjustable rate of injection or withdrawal or constant rate control. Furthermore, on the tope of the air compressor there is a pressure sensor, which allows detection of holes or leakage on the balloon 12 through the pressure variation in the balloon 12 when compressed air is charged into the balloon 12. Hence, when air compressor is operated to suck or deliver air, whether there is clogged injection outlet or leakage can be determined by the internal pressure variation in the balloon 12. When special conditions arise (such as patients in coma or balloon rupture . . . etc.), the air compressor power can be immediately switched off to interrupt the withdrawal or injection action. When blood withdrawal is performed on artery, first the balloon 12 is inflated to contact the inner wall of the syringe body 10, and then the injection needle along with the needle cap 16 is placed on the needle socket 11. Afterward, when the needle cap 16 is pressed onto the patient skin, the pressure sensor will read different variation on artery than normal blood vessels. At this moment, the needle cap 16 is removed for blood withdrawal on artery. The pressure sensor can still read the signal when the needle is inserted into the artery, so the medical staffs are guided to continue the blood withdrawal. Further, the air compressor has an output/input device that is connectable to computer, PDA or mobile phone. Furthermore, the air compressor can be replaced by hydraulic pump. Alternatively, the air compressor can have a transport tubing 15 to connect to an inflatable thin pad, which can be wrapped around the upper place of the injection position. When the thin pad is charged with air from the air compressor, it inflates to replace the traditional wrapping rubber band.

Please refer to FIG. 34. The rear receptacle 142 of the nozzle 14 can be replaced by a penetration section 143. The outer edge of the penetration section 143 extends backward to form a slope, on which there are several locking bulging blocks 1431 to allow tight match between the bulging blocks 1431 and the inner wall of the transport tubing 15 when the tubing 15 is inserted into the penetration section 143.

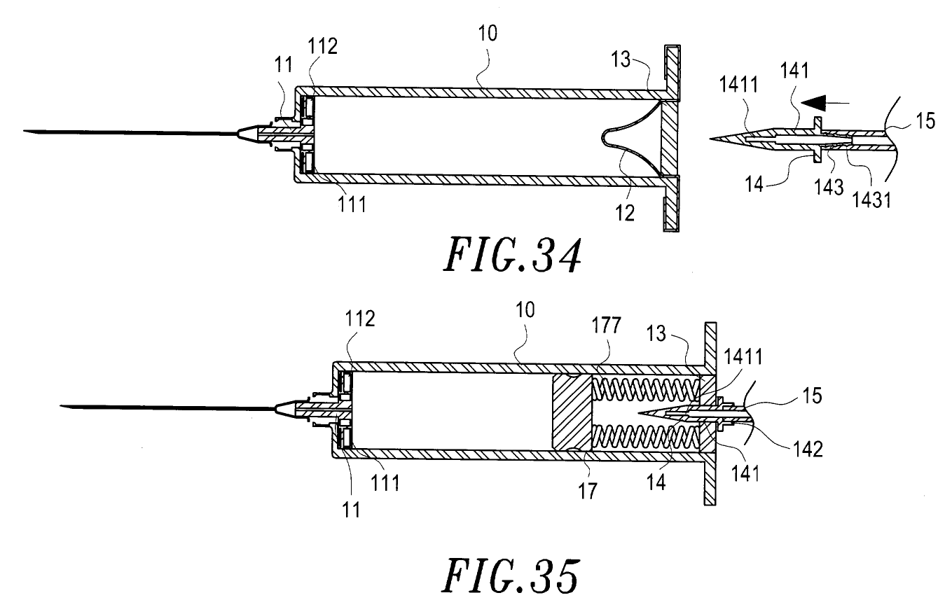

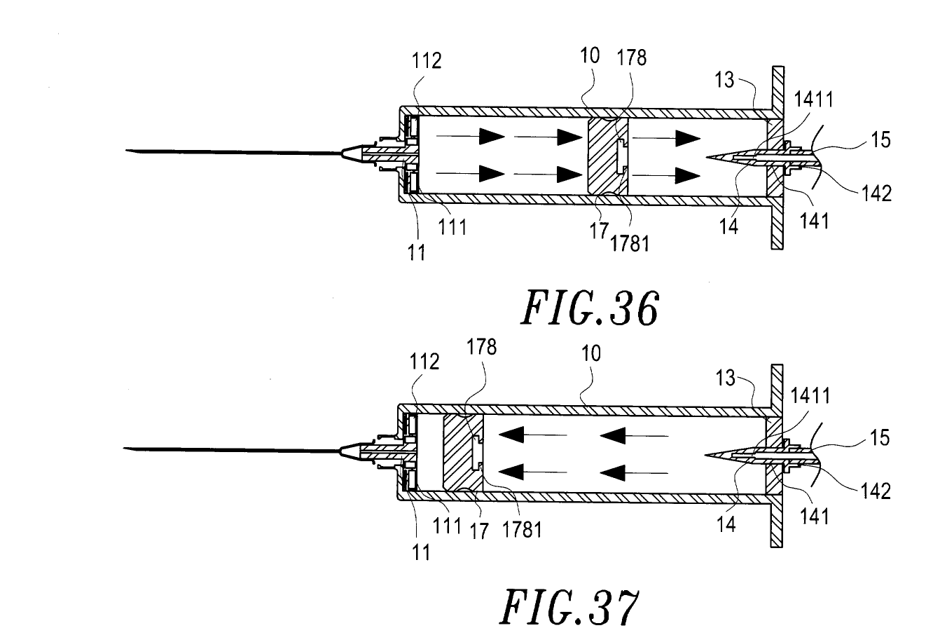

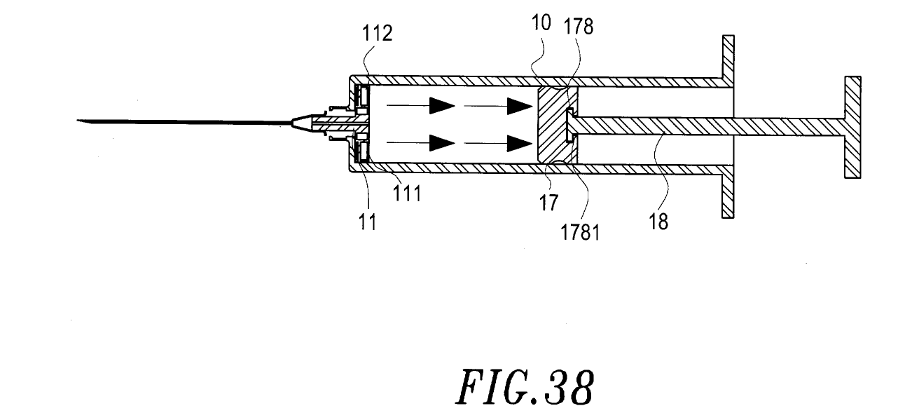

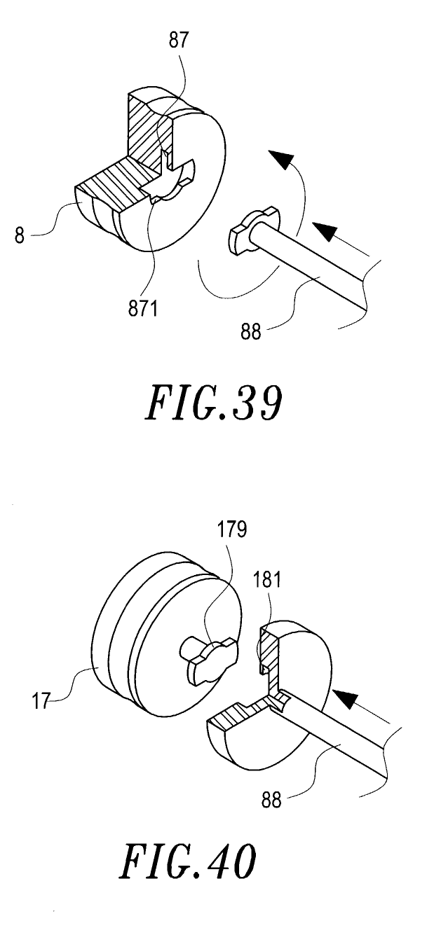

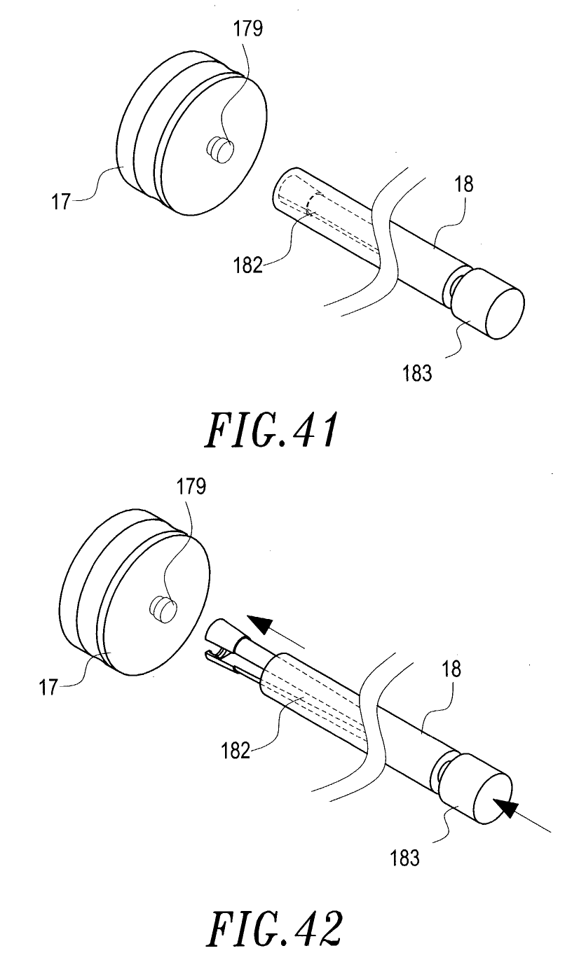

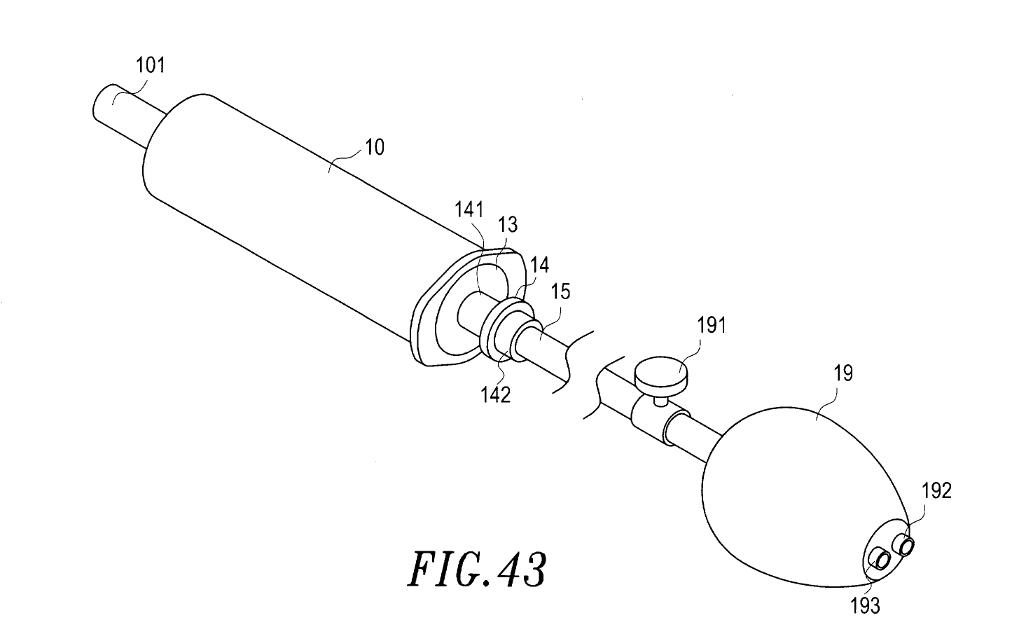

Please refer to FIGS. 35, 36, 37, 38, 39, 40, 41, 42 and 43. A piston 17, which outer edge fits the inner wall of the syringe body 10 tightly, can replace the balloon 12 of the needle syringe in the present invention. So an airtight chamber is formed between the piston 17 and the plunger 13. The rear of the piston 17 can connect to one or more than one elastic elements 177. One end of the elastic element 177 connecting to the piston 17 can be connected to the inner wall of the syringe body 10 or the plug 13. The piston 17 is placed into the syringe body 10 through the opening at the rear. When the needle syringe for the present invention is in use, the needle is inserted into drug bottle. Air compressor will suck out the air in the closed airtight chamber. So the chamber is in a negative pressure. The plunger will pull the piston 17 backward to create vacuum suction force between the syringe body 17 and the needle socket 11. The drug in the drug bottle is withdrawn into the syringe body 10 through the needle and stays in the space between the needle socket 11 and the piston 17. Then, the needle syringe is inserted into patient body. Air is pumped into the syringe body 10 by air compressor to create a pushing force that the piston 17 moves the drug toward the needle head. The piston 17 helps injection of the drug in the syringe body 10 through the needle head to the patient body. In addition, at the rear of the piston there can be a holding groove 178, which has several bulging blocks 1781 at the opening. When power failure occurs or air compressor faults, the bulging blocks 1781 are pushed by the plunger 18 into the holding tank and secured by rotation. The piston plunger 18 is used to replace the air compressor to move the piston forward or backward. Further, at the rear of the piston 17 there can be a fastener 178 and at the front of the piston 17 there can be a fixation groove 181 for the fastener 178. Or inside the piston plunger 18 there can be a pressure-retainer 182, which has a press 183 at the rear of the piston plunger 18. Through forcing the press 183, the pressure-retainer is held by the fastener 178 of the piston 17. Again, when power failure occurs or air compressor faults, manual compressor 19 can be used. There is an exhaust valve 191 at the connection of the manual compressor 19 and the transport conduit 15. At the rear of the manual compressor there are a unidirectional inlet valve 192 and a unidirectional exhaust valve 193.

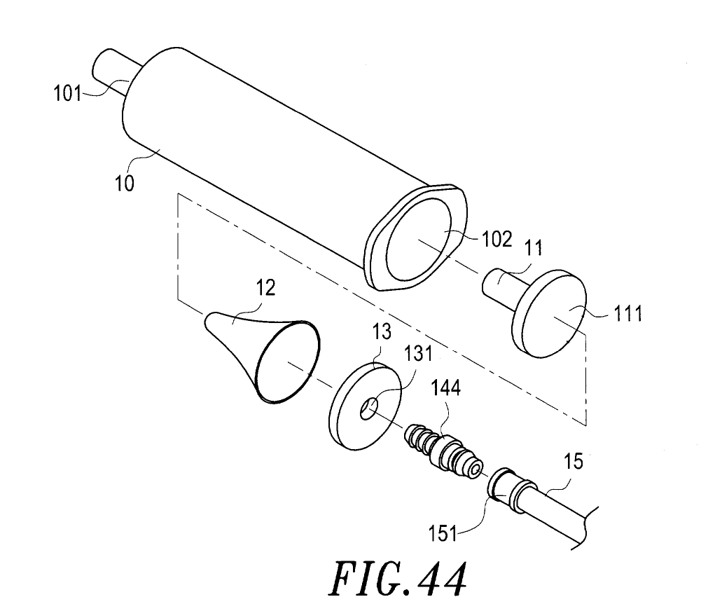

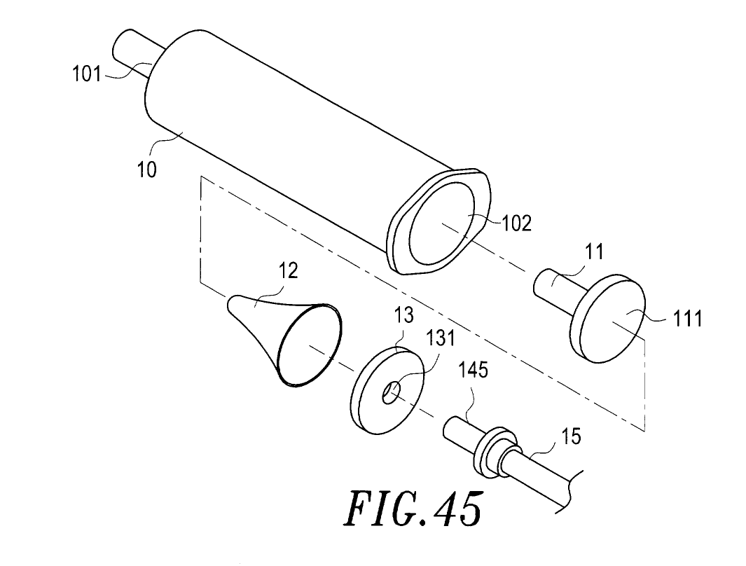

Please refer to FIGS. 44 and 45. The cylindrical plug 13 for the present invention can also be made of non-elastic material. Besides, on the cylindrical plug 13 there are vent holes 131 and a quick connector 144 is used to replace the nozzle 14. On the transport tubing 15, there is a connector base 151 to connect to the quick connector 144. Further, a normal connector 145 can replace the said quick connector 144. The transport tubing 15 then connects to the rear of the normal connector 145.

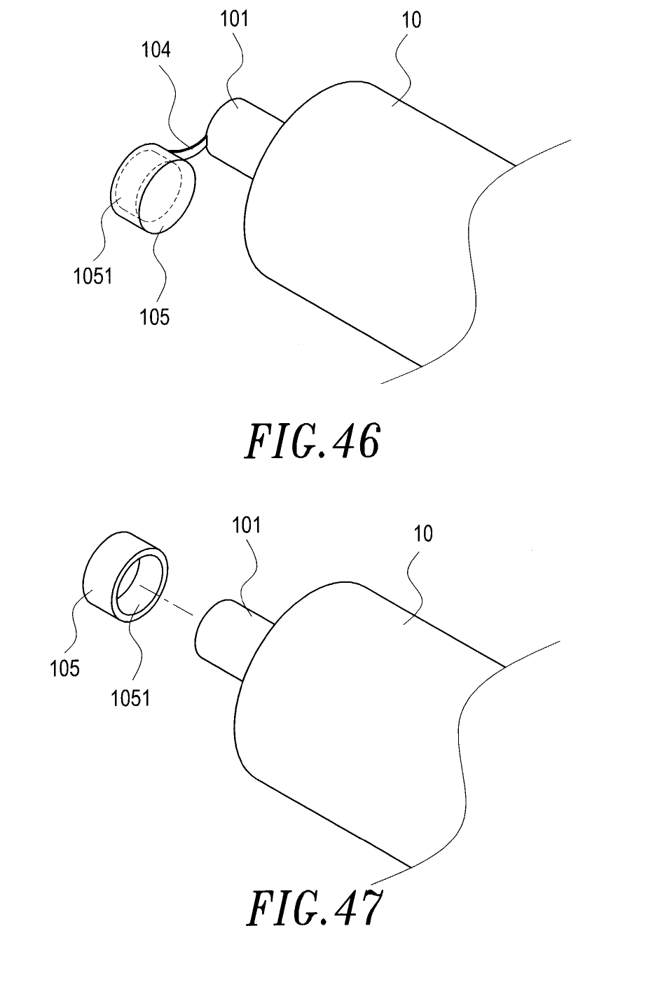

Please refer to FIGS. 46, 47 and 48. At the front outer edge of the syringe body 10 there cab be an elastic connection end 104, the other end of which connects to a cap, inside which there is a receptacle 1051 installed at the front outer edge of the cap 105. Further, the cap 105 does not need to connect to the syringe body 10. The receptacle 1051 in the cap 105 can be replaced by a bulging holder 1052, which can be placed in the needle connector 11 at the front of the syringe body 10.

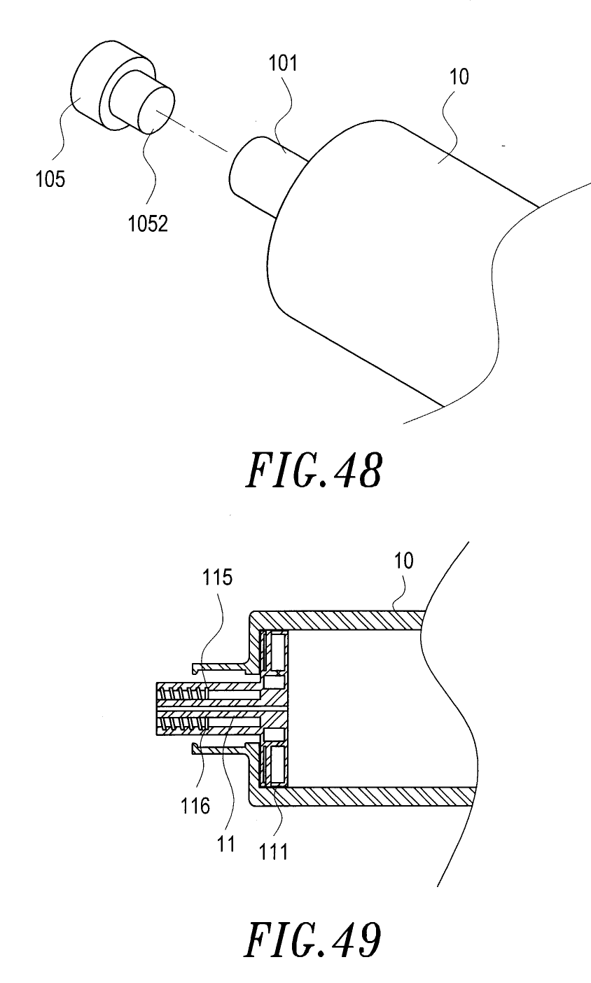

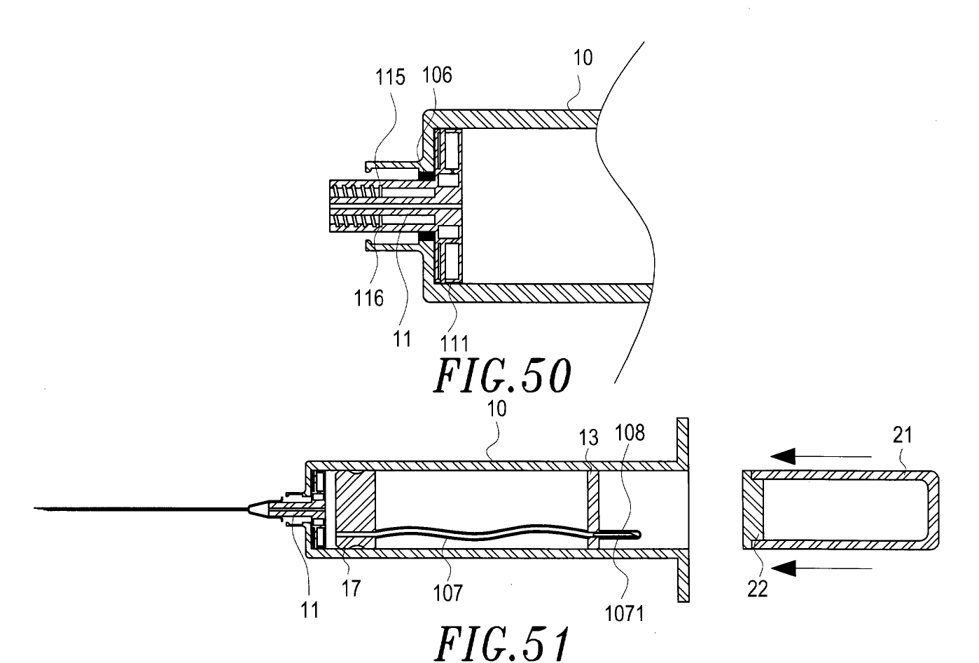

Please refer to FIGS. 49 and 50. At the outer edge of the needle socket 11 there is an exterior ring 115, which has screw threads 116 for tightening the needle. In addition, the rear of the retainer 1011 has an elastic element 106, which holds onto the outer edge of the exterior ring 115. When the needle socket 11 retracts into the syringe body 10, the elastic element 106 bounces to the original position of the exterior ring 115, so the needle socket 11 can not return to the original position due to blockage by the elastic element 115. When the elastic element 106 bounces back to the original position of the exterior ring 115, the front of the needle cap 106 can be placed in the middle of the elastic element 106 to seal the injection needle into the syringe body 10.

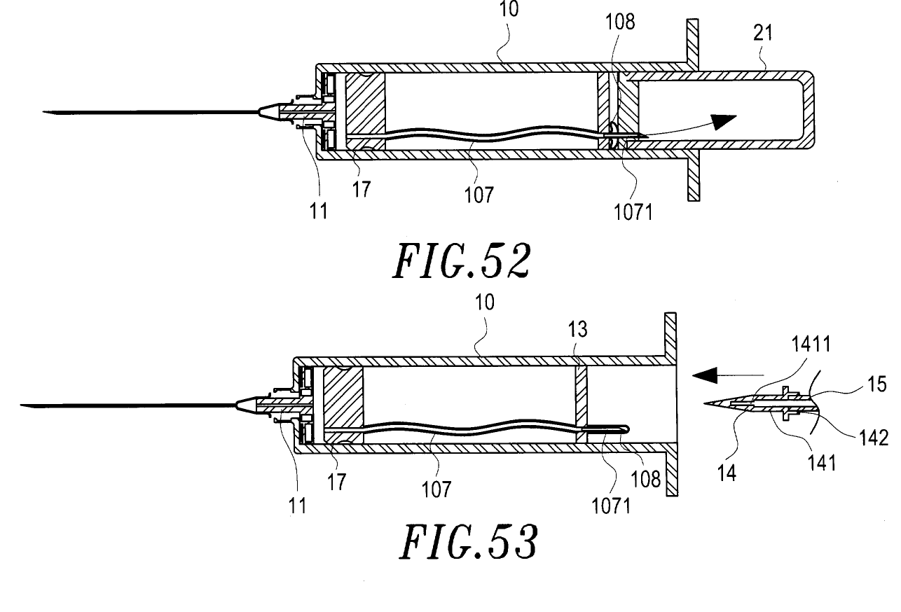

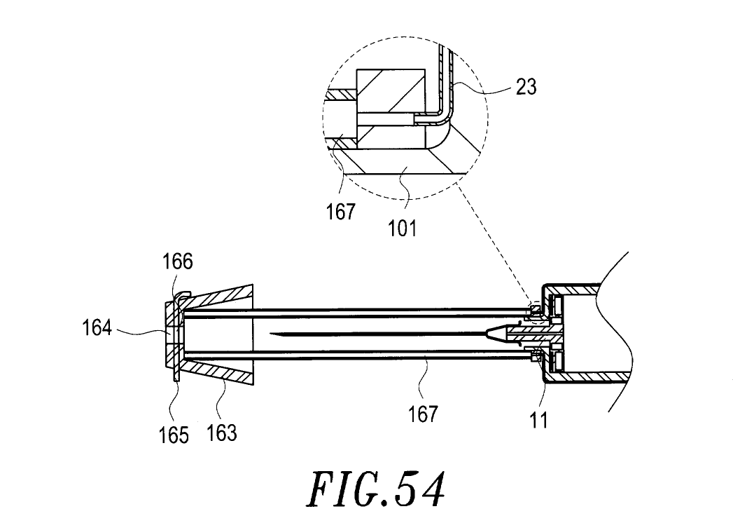

Please refer to FIGS. 51, 52 and 53. The balloon 12 for the present invention can be replaced by a piston 17, between which and the cylindrical plug 13 there is a soft tubing 107, one end of which connects to the piston 12 and passes through the piston 12, while the other end of which passes through the cylindrical plug 3 and connects to a needle head 1071, which outer edge has an exterior cover 108 made of compressible materials. When blood sampling is conducted, it only needs to push a test tube 21 into the syringe body 10 and allow the needle head 1071 to penetrate the soft cork 101 of the test tube 21 and punch the exterior cover 108. In the meantime, due to negative pressure in the test tube 21, the blood is withdrawn through the soft tubing 107 directly from the needle to the test tube 21. When repeated blood sampling is required, there is no need to remove the needle from the patient body, but to replace the test tube 21. When the blood sampling is completed, the nozzle 14 is inserted into the cylindrical plug 13 and air is charged into the syringe body 10 through the nozzle 14. Hence, the air pushes the piston 107 to break up the connection between the needle socket 11 and the syringe body 10. Finally, the needle socket 11 along with the injection needle retracts into the syringe body 10.

Please refer to FIG. 54. The soft cork 101 is for the placement of a needle head cap 16. The needle head cap 16 can connect to a needle cap 163 via an inflation jacket 167. A penetration hole 164 for syringe is made on the needle cap 163. Besides, a retainer 165 is also made on the needle cap 163. Several round holes 166 are made on the retainer 165. Further, at the fringe for the syringe body 10 there are several inflation conduits 167, which connect to an air compressor at the other side. When the needle syringe for the present invention is in use, the air compressor will suck out the air inside the inflation conduits 167 at the needle head cap 16 and push back the needle cap 163. The needle head will stick out of the needle cap 163 through the penetration hole 164 and the round holes 166. The needle cap 163 is fixed onto the needle head. When injection or withdraw is completed, the air compressor will fill air into the inflation jacket 167 and generate a pushing force to push out the needle cap 163 out of the needle head. Meanwhile, the needle socket is pulling the needle head into the syringe body 10. With the forces in opposite directions, the needle cap 163 is separated from the needle head. Besides, the inflation jacket 167 is filled with air by the air compressor to obtain certain strength and cover up the front edge of the syringe body 10. It also pulls the retainer 165 to cover up the needle hole 164 on the needle cap 163, so the needle head is prevented from sticking out of the needle hole 164 and hurting people.

By comparing with the above-mentioned methods or other traditional methods, the needle syringe for the present invention has the following advantages:

1. For the needle syringe for the present invention, an air compressor is used to make a balloon to push drugs or blood in the syringe body to a test tube, patient body or a test tube for blood storage through an injection needle.

2. For the needle syringe for the present invention, an air compressor is used to suck air out of the balloon from a nozzle through a transport tubing and form vacuum suction inside the syringe body, so blood or drugs can be withdrawn from the patient body or a drug bottle into the syringe body through the needle socket.

3. For the needle syringe for the present invention, a display or an alarm connecting to an air compressor will sound to notify medical staffs when injection or withdrawal is completed or clogged injection outlet or leakage is found.

4. For the needle syringe for the present invention, a controller, a decoder and a flow meter connecting to an air compressor are used to adjust the injection or withdrawal rate and to control timing and constant quantity.

5. For the needle syringe for the present invention, a pressure sensor connecting to an air compressor is used to determine whether a hole or leakage occurs in the balloon through the detection of pressure variation in the balloon when air is compressed into the balloon by the air compressor. Similarly, clogged injection outlet or leakage can be determined by pressure variation in the balloon when the air compressor is sucking or charging air.

6. For the needle syringe for the present invention, the power switch for the air compressor can be used to stop the action of withdrawal or injection.

7. For the needle syringe for the present invention, an output/input device on the air compressor is available for connection with computer, PDA or mobile phone.

8. For the needle syringe for the present invention, the elastic fixture in the blocking section can be loosen by pressing the blocking section of the needle socket, so the needle socket along with the injection needle can retract into the syringe body and medical staffs or patients can be protected from infection due to needle stick.

9. For the needle syringe for the present invention, a locking tenon in the needle cap is locked with the retainer of the needle connector on the syringe body to prevent dangers due to injection needle sticking from the syringe body.

10. For the needle syringe for the present invention, destroying the connection between the needle socket and the syringe body can not reuse the needle.

11. For the needle syringe for the present invention, the artery injection position can be located because the pressure sensor on the air compressor can read the signal variation when the syringe is pressed onto the patient skin, so medical staffs can be guided for blood withdrawal on artery.

12. For the needle syringe for the present invention, the balloon is replaced by a piston. A soft tubing is installed between the piston and the cylindrical plug. One end of the soft tubing connects to a needle head for repeatable blood sampling.

Many changes and modifications in the above-described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A needle syringe, using an air compressor to suck air out of said needle syringe and creating vacuum suction for withdrawal of drugs or blood, or using an air compressor to charge air into said needle syringe so push force generated to inject drugs into patient's body.

2. The needle syringe of claim 1, wherein said needle syringe comprises a syringe body, a needle socket, a balloon, a cylindrical plug, a nozzle and a transport tubing, with a needle connector at one end and an opening at the other end.

3. The needle syringe of claim 1, wherein an air compressor of said needle syringe connects to a display and an alarm, said alarm will sound to notify medical staffs when injection and withdrawal are completed or clogged injection outlet or leakage is found.

4. The needle syringe of claim 1, wherein said air compressor of said needle syringe connects to a controller, a decoder and a flow meter, so an injection or withdrawal rate can be adjusted and time control and constant rate can be operated.

5. The needle syringe of claim 1, wherein said air compressor of said needle syringe can be replaced by a manual air compression structure, said manual air compression structure has an exhaust valve at a connection with a transport tubing and has an unidirectional inlet valve and an unidirectional exhaust valve at a rear.

6. The needle syringe of claim 1, wherein said air compressor of said needle syringe has a pressure sensor, said pressure sensor can detect pressure variation in said balloon and whether rupture or leakage exists in said balloon when air is charged into said balloon, so whether said injection outlet is clogged or has leakage can be determined when said air compressor is sucking or compressing air, and similarly said pressure sensor can read artery pulses to guide medical staffs to perform blood sampling when said needle syringe is pressed onto patient's skin.

7. The needle syringe of claim 1, wherein said air compressor of said needle syringe has a power switch, said power switch is switched off to interrupt withdrawal or injection action.

8. The needle syringe of claim 1, wherein said air compressor of said needle syringe has an output/input device connectable to computer, PDA or mobile phones.

9. The needle syringe of claim 1, wherein a hydraulic pump can replace said air compressor of said needle syringe.

10. The needle syringe of claim 1, wherein said air compressor of said needle syringe has an inflation thin pad connecting to said transport tubing to replace a rubber band tied on an upper place of an injection position.

11. The needle syringe of claim 2, wherein an inner edge of said needle connector of said needle syringe has a concave retainer.

12. The needle syringe of claim 2, wherein said needle socket is installed inside said syringe body of said needle syringe and a socket is available at the front and a blocking section is at the rear to have a holding space for an elastic fixture, said elastic fixture can be loosened off by pressing said blocking section to make said needle socket and said needle retract into said syringe body.

13. The needle syringe of claim 2, wherein said balloon of said needle syringe is in a stripe of elastic materials and installed at an outer edge of the rear of said syringe body with non-external part placed in said syringe body.

14. The needle syringe of claim 2, wherein said cylindrical plug of said needle syringe is a cylinder of elastic materials and placed at an opening of the rear of said syringe body.

15. The needle syringe of claim 2, wherein a front of said nozzle of said needle syringe has a socket for said plug to pass through said nozzle into said balloon and several vent holes are on said socket, the rear of said nozzle has a receptacle, said receptacle has a transport tubing, besides, the other end of said transport tubing connecting to said nozzle is installed on said air compressor.

16. The needle syringe of claim 2, wherein said balloon of said needle syringe can be replaced by a piston, an outer edge of said piston fits tightly with an inner wall of said syringe body, so an air-tight closed chamber is formed between said piston and said cylindrical plug, Further, the rear of the piston can connect to one or more than one elastic elements. Another end of the connection of the elastic element and the piston can connect onto the inner wall of the syringe body or the cylindrical plug.

17. The needle syringe of claim 2, wherein a front outer edge of said needle syringe can have an elastic connection end, the other end of said elastic connection end connects to a cap, said cap has a concave holding space that can fit the needle connector at a front of said syringe body.

18. The needle syringe of claim 2, wherein said balloon can be replaced by a piston, between said piston and said cylindrical plug there is a soft tubing, one end of said soft tubing connects to said piston and passes through said piston, while the other end of said piston passes through said cylindrical plug to connect a needle head, at an outer edge of said needle head a cover of compressible material is installed.

19. The needle syringe of claim 2, wherein the soft cork is for placement of the needle cap, the needle head cap can connect to a needle cap through inflation, the needle cap has a needle hole and a retainer, the retainer has a round hole; further, at the fringe of the syringe body there are several inflation conduits with one side connecting to the inflation jacket and the other side to the air compressor.

20. The needle syringe of claim 11, wherein the rear of a retainer of said syringe body has a wrapped elastic element to bounce to a needle socket position when said needle socket retracts into said syringe body.

21. The needle syringe of claim 11, the inner edge for the needle cap of the needle syringe has threads for the bulging part to screw in.

22. The needle syringe of claim 12, wherein a circular elastic device is installed between said syringe body and a blocking section of said needle socket to increase resistance for said blocking section.

23. The needle syringe of claim 12, wherein said blocking section does not need a holding space for said elastic fixture, said blocking section can be made of elastic materials to have its outer edge contact with an inner wall of said syringe body, while said balloon is replaced by a piston, at an outer edge of said piston corresponding to said blocking section there are locking hooks.

24. The needle syringe of claim 12,inside the retainer of the needle syringe the holding space of the elastic fixation can be omitted. Instead, an air bag made of balloon material is used at the rear of the retainer. The outer edge of the air bag extends to cover the outer side of the retainer. At the rear of the air bag, there is one or more than one air conduits made of elastic material. Besides, there are several bulging parts on the inner wall of the syringe body.

25. The needle syringe of claim 12, wherein said blocking section of said needle syringe may not need a holding space for said elastic fixture, however, at an outer edge of said blocking section there is a spring coil, in addition, between said syringe body and said blocking section there is one or more than one support, inside said syringe body, there is a piston, at one side of said piston corresponding to said spring coil a bulging block exists at an outer edge of said piston to form a clamping section, a top inner edge of said bulging block has a spherical bulging part.

26. The needle syringe of claim 12, wherein said blocking section of said needle syringe may not need a holding space for said elastic fixture, however, the rear of said blocking section has several locking tenons with reversed hooks, and said syringe body has a piston of elastic materials inside.

27. The needle syringe of claim 12, wherein said blocking section of said needle syringe may not need a holding space for said elastic fixture, however, the rear of said blocking section has several circular grooves, at an outer edge of said balloon corresponding to said circular groove there are several spherical bulging parts.

28. The needle syringe of claim 12, wherein said elastic fixture of said needle syringe is composed of two interlocking pushing stoppers, a bulging block is on an extension of one stopper, said stopper is on an outer edge of said blocking section and holds onto an inner wall of said syringe body, and said two pushing stoppers adhere with each other by adhesive.

29. The needle syringe of claim 12, wherein said needle socket of said needle syringe is installed at an outer edge of said injection needle and can be equipped with an external ring, said external ring has screw threads inside for injection needle to screw in.

30. The needle syringe of claim 14, wherein said cylindrical plug of said needle syringe can be made of non-elastic material, said cylindrical plug has a penetration hole and uses a quick connector to replace said nozzle, there is a connection base on said transport tubing for connection of a quick connector; besides, a fastener can be set at the rear of the piston. The front of the piston plunger can have a fixation groove for the fastener. Further, inside the plunger, there can be a pressure retainer, which press is at the rear of the piston plunger.

31. The needle syringe of claim 15, wherein a penetration section can replace said receptacle at the rear of said nozzle of said syringe, an outer edge of said penetration section extends outward to form a slope, said slope has several locking bulging blocks to fit tightly with an inner wall of said transport tubing when said transport tubing is inserted in said penetration section.

32. The needle syringe of claim 16, wherein said balloon of said syringe can be replaced by a piston, the rear of said piston has a fixing groove, said fixing groove has several holding blocks at an opening and is used for a plunger to push in and lock in by rotation.

33. The needle syringe of claim 17, wherein said concave holding space of said cap of said syringe can be replaced by a bulging holder, said bulging holder holds onto a needle connector at a front of said syringe body.

34. The needle syringe of claim 17, wherein said cap of said syringe does not need to connect to said syringe body.

35. The needle syringe of claim 23, wherein said piston of said syringe can be replaced by a balloon, at a front of said balloon there is a circular bulging part.

36. The needle syringe of claim 24, wherein said spring coil of said syringe can be made of non-elastic materials.

37. The needle syringe of claim 25, wherein said piston of said syringe can be made of non-elastic materials, there are several guiding holes in corresponding quantity and at corresponding position to a locking tenon, a wider holding groove is installed at a bottom of each guiding hole, inside said guiding groove, there is a pathway connecting to said needle socket for drug passage.

38. The needle syringe of claim 25, wherein said blocking section of said needle syringe can be made of elastic materials, said piston has several locking tenons with reversed hooks at one side corresponding to said blocking section.

39. The needle syringe of claim 28, wherein said elastic fixture of said needle syringe can be composed of one or more than one T stoppers and several springs, one end of said spring has a bulging block, said bulging block extends along an outer edge of said blocking section and holds onto an inner wall of said syringe body, the other end of said spring holds onto a longer side of said T stopper, at one side of said holding space, there is a groove for placing a longer extension side of said T stopper.

40. The needle syringe of claim 28, wherein said elastic fixtures can be held onto said holding space of said blocking section by several springs, so two sides of said blocking section hold onto an inner wall of said syringe, said holding space has a push block at one side of each spring.

41. The needle syringe of claim 28, wherein said elastic fixture can be composed of several clamping modules and a spring coil of rubber-like elastic materials, an outer edge of said spring coil holds onto an inner wall of said syringe body and an inner side of said coil spring holds onto each clamping module.

42. The needle syringe of claim 30, wherein said quick connector of said syringe can be replaced by a normal connector, so said transport tubing connects to a rear of said normal connector.

43. The needle syringe of claim 38, wherein said blocking section of said syringe can be made of non-elastic materials, several guiding holes are open at a corresponding position and in a corresponding quantity to said locking tenons, a wider holding groove is installed at a bottom of each guiding hole, a pathway in said holding groove can be open to connect to said needle socket for drug passage.

44. The needle syringe of claim 41, wherein said clamping module of said syringe is made of a triangular elastic plate, two sides of an opening of a triangular elastic plate are bent into a spherical clamping section, inside said spherical clamping section, there is a lever that is extended to an outer edge of said triangular elastic plate, an arch elastic structure is formed at a bottom of said triangular elastic plate.
